(19) United States
(12) Patent Application Publication
(10) Pub. No.: US 2005/0197597 A1
(43) Pub. Date: Sep. 8, 2005

(54) Guidewire with hollow distal section

(75) Inventor: Nareak Douk, Lowell, MA (US)

Correspondence Address: Catherine C. Maresh;Medtronic Vascular, Inc., 3576 Unocal Place, Santa Rosa, CA, 95403, US

(73) Assignee: MEDTRONIC VASCULAR, INC.

(21) Appl. No.: 10/794,530

(22) Filed: Mar. 5, 2004

(51) Int. Cl. A61B 5/00

(52) U.S. Cl. 600/585

(57) ABSTRACT

A medical guidewire includes a proximal section, a hollow distal section and a joint there between. The hollow distal section has decreasing wall thickness along a portion of its length.

### FIELD OF THE INVENTION

This invention relates generally to medical guidewires, and particularly to the design and fabrication of metallic guidewires having a proximal section with relatively high columnar strength, and a hollow distal section with increasing lateral flexibility in a distal direction along its length.

### BACKGROUND

Cardiovascular disease, including atherosclerosis, is a leading cause of death in the U.S. A number of methods and devices for treating coronary heart disease have been developed, including a broad array of catheters and guidewires and minimally invasive methods for using them. Catheter-based delivery systems are routinely used to introduce stents and other medical devices into the cardiovascular system for both therapeutic and diagnostic purposes. Many of the catheters used in such delivery systems, including over-the-wire catheters and rapid exchange catheters, require a guidewire to direct the catheter through the vascular system.

Typically, the guidewire is inserted into the vascular system through a needle puncture in an artery, such as the femoral, jugular, or subclavian artery. The guidewire is then threaded through the vascular system until the distal end of the guidewire is adjacent to the treatment site. The distal portion of the catheter is slipped over the proximal end of the guidewire, and the catheter is advanced along the guidewire through the vasculature until the distal portion of the catheter is positioned at the target site. The position of the catheter end may be determined by common visualization methods such as fluoroscopy or ultrasound.

Guidewires may also be used as a core member to construct low profile intravascular devices such as so-called “balloon-on-a-wire” or fixed-wire dilatation catheters, filter guidewires and occluder guidewires. Medical procedures using catheters that require guidewires include percutaneous transluminal angioplasty, stent delivery, atherectomy, treatment of aneurysms and other catheterization procedures.

In order to perform well, a guidewire must have sufficient columnar strength and rigidity so that it can be pushed through the vasculature of the patient without bending back on itself or kinking. However, if it is too stiff, it may cause damage to blood vessel walls. At the same time, the guidewire must be sufficiently flexible so that it can follow a winding, sometimes tortuous, path through the patient's vasculature. In order to balance the need for both flexibility and columnar strength, guidewires are frequently constructed to have a relatively rigid proximal section and a more flexible distal section. Such a balanced combination also provides a guidewire with good steerability, which is the ability to transmit substantially all rotational inputs from the proximal end to the distal end.

Available guidewires for vascular procedures generally include an elongated core member having one or more tapered sections near the distal end of the core member and a helical coil or a polymer jacket surrounding the tapered distal section of the guidewire. Typically, the outer diameter of the helical coil or polymer jacket is the same as the outer diameter of the proximal section of the guidewire so that the exterior of the guidewire presents a smooth surface of constant diameter. However, the distal section of the guidewire is increasingly flexible because of the reduced diameter of the inner core member. The helical coil or polymer jacket does little to reduce the lateral flexibility of the distal section.

One such guidewire is disclosed in U.S. Pat. No. 6,142,975 and comprises a metallic inner core element, two metallic layers surrounding the inner core element and a flexible outer body such as a helical coil. The composition and thickness of each layer are chosen to produce the desired flexibility or rigidity in each region along the length of the guidewire. In one embodiment, the distal section of the guidewire comprises a distally tapered, stainless steel core member, a first outer layer comprised of a pseudoelastic alloy such as nitinol, and a second outer layer of stainless steel. This arrangement provides both the desired mechanical properties and stainless steel surfaces that are readily bonded or soldered for attachment of the various guidewire components.

U.S. Pat. No. 6,575,920 discloses a guidewire having an elongated inner member situated within the lumen of a tubular outer member. The diameter of the inner member may be tapered so that the distal section of the inner member is more flexible than the outer member, and the distal end of the inner member extends beyond the tubular outer member. This configuration allows the distal end of the guidewire to flex laterally until the inner member engages the distal section of the wall of the outer member. The degree of flexibility is controlled by the length of the inner member that extends beyond the outer tubular member.

U.S. Pat. No. 6,592,570 and U.S. Pat. No. 6,638,372 disclose a superelastic guidewire that is composed of nickel, titanium, and platinum or another element. The composition of the ternary alloy is selected so that the guidewire is capable of undergoing a stress-induced phase change at body temperature and thus giving the guidewire both the columnar strength and the flexibility needed during use in the body.

One difficulty frequently encountered in the manufacture of guidewires is joining two metallic sections to each other. For example U.S. Pat. No. 6,592,570 discloses coupling proximal and distal sections of a guidewire by placing a tightly fitting tube over the ends of the two sections and securing the ends to the interior surface of the tube. Both U.S. Pat. No. 6,592,570 and U.S. Pat. No. 6,645,159 describe forming a tightly fitting junction between two segments of a guidewire by forming a rod-shaped portion on the end of one segment and a complementary tube-shaped portion on the end of the adjacent segment. The rod may be inserted into the tube to form a male/female junction and held in place by crimping, swaging, welding, soldering, or applying an adhesive to secure one to the other.

Many guidewires include, at the distal end of the guidewire, a shapeable member that can be manually formed by the physician at the time of use. U.S. Pat. No. 6,645,159 discloses a distal shapeable member that may be the distal end of the tapered inner core of the distal section of the guidewire or it may be a separate shaping ribbon attached to the distal end of the guidewire. U.S. Pat. No. 6,592,570 discloses a distal shapeable member formed by flattening the distal end of the inner core of the distal section of the guidewire. This design eliminates the need for a weld or some other means of securing the shaping member to the distal end of the guidewire. However, it requires that the metal alloy or other material of the inner core be appropriate for the shapeable member as well.

The guidewires disclosed in each of these patents are complex in design and require precise and costly construction methods. It would be desirable, therefore, to provide a guidewire that has a simplified design, is inexpensive to manufacture, has the desired physical characteristics, including columnar strength of a proximal section and increasing lateral flexibility of a distal section.

### SUMMARY OF THE INVENTION

One embodiment of the invention provides an intravascular guidewire comprising an elongated member having a proximal section and a hollow distal section. The distal section of the guidewire has a decreasing wall thickness along at least a portion of its length.

Another embodiment of the invention provides an intravascular guidewire comprising an elongated member having a proximal section and a hollow distal section in which the distal section includes means for providing increased lateral flexibility along the length of the distal section.

The present invention is illustrated by the accompanying drawings of various embodiments and the detailed description given below. The drawings should not be taken to limit the invention to the specific embodiments but are for explanation and clarity. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings, which are not to scale.

### BRIEF DESCRIPTION OF THE DRAWINGS

FIG. 1 shows a lateral view of a portion of a guidewire in accordance with the present invention;

FIG. 2 depicts longitudinal cross-section of a hollow distal section of a guidewire in accordance with the present invention;

FIG. 3 depicts a longitudinal cross-section of a distal portion of a guidewire assembly in accordance with the present invention;

FIG. 4 shows an exploded view, in longitudinal cross-section, of a distal portion of a guidewire in accordance with the present invention;

FIG. 5 shows, in longitudinal cross-section, an assembly of guidewire components illustrated in FIG. 4;

FIG. 6 depicts a longitudinal cross-section of a distal portion of an alternative guidewire embodiment in accordance with the present invention; and

FIG. 7 shows a partially-sectioned lateral view of a portion of a guidewire assembly having a flexible sleeve in accordance with the present invention.

### DETAILED DESCRIPTION

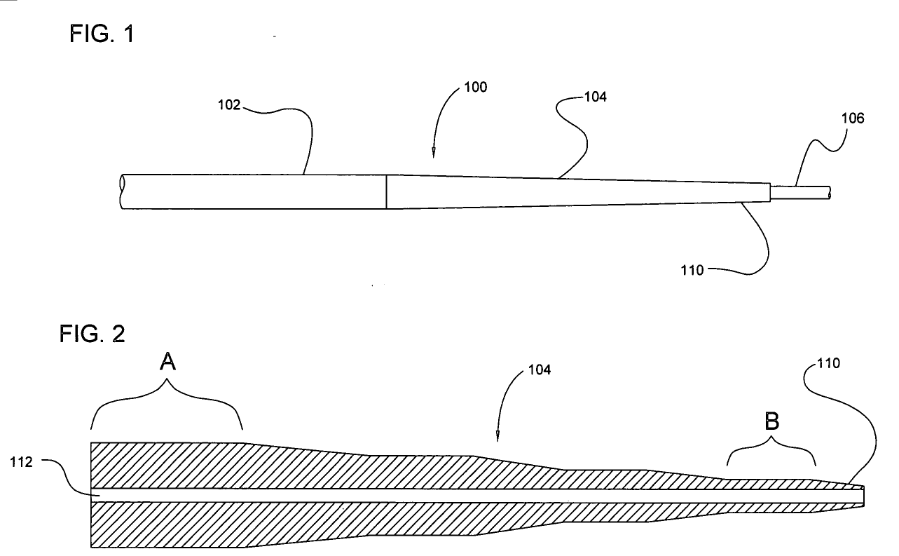

Throughout this specification, like reference numbers refer to like structures. FIG. 1 shows guidewire 100 including proximal section 102, hollow distal section 104 and shapeable member 106 extending from distal end 110 of distal section 104. Proximal section 102 has a high columnar strength sufficient to enable the guidewire 100 to be pushed through a patient's vascular system or other body lumen without kinking. Proximal section 102 may comprise one or more metals selected from stainless steel, titanium, MP35N cobalt-based alloy, nickel titanium (nitinol), tungsten, or other bio-compatible rigid or semi-rigid material. The length of proximal section 102 is selected according to the intended use of guidewire 100. In one embodiment of the invention, the diameter of proximal section 102 may be up to about 0.030 inches. However, the diameter of proximal section 102 will be as small as required to fit slidably within suitable catheters or other medical devices. In one embodiment of the invention, the outer diameter is approximately 0.014 inches.

Hollow distal section 104 is attached to distal end 108 of proximal section 102. The outer diameter of distal section 104 decreases in a distal direction along the length of distal section 104. The outer diameter at the proximal end of distal section 104 is typically selected to be similar to the diameter of proximal section 102 such that a joint between sections 102 and 104 can have a relatively smooth, continuous, external surface. Shapeable member 106 is attached to distal end 110 of distal section 104.

FIG. 2 illustrates a longitudinal cross-section of hollow distal section 104 of guidewire 100. Inner bore 112 extends longitudinally through distal section 104 and may have a constant diameter. In one embodiment of the invention, the diameter of inner bore 112 is between 0.002 and 0.003 inches. Since hollow distal section 104 has a through-bore, it may also be described as being tubular, though the wall thickness of distal section 104 varies along its length. Distal section 104 may be made from heavy-walled, also called thick-walled, metal tubing. In one embodiment of the invention, the outer surface of a heavy-walled metal tube is selectively removed, as by centerless grinding, so that the outer diameter is tapered along its length. The wall thickness of distal section 104 may be selectively varied using other methods such as acid or laser etching or rotary swaging.

As wall thickness decreases along the length of distal section 104, lateral flexibility increases. The extent of the taper of each region of distal section 104 is selected to produce the desired flexibility in that region. For example, region A in FIG. 2 has greater wall thickness and correspondingly less flexibility than region B. The term “taper” herein refers to any reduction in diameter over a length. Such a reduction in diameter may have a constant rate or angle, or it may comprise a series of regions having different taper angles. Tapered regions may also abut, or may be interposed between, constant-diameter cylindrical regions.

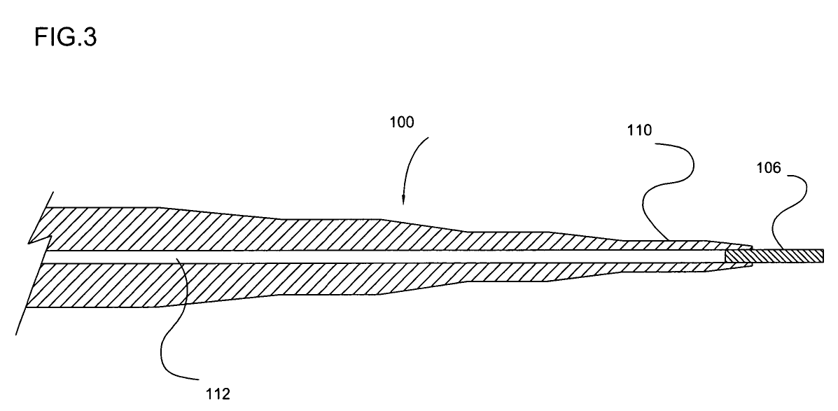

FIG. 3 shows hollow distal section 104 of guidewire 100 with shapeable member 106 attached to distal end 110 of section 104. Shapeable member 106 is a metallic ribbon or wire that may be manually formed by the physician to create a bent, steerable tip, as is known by those of ordinary skill in the art of guidewires. Shapeable member 106 may have, for example, a diameter of 0.002 to 0.003 inches and may comprise stainless steel or other biocompatible materials that provide the desired combination of strength, flexibility, and ductility for shaping. Shapeable member 106 may be coupled to distal end 110 of distal section 104 by inserting the proximal end of shapeable member 106 into inner bore 112 of distal section 104 to provide a concentric joint that does not require an additional coupling member. The portion of shapeable member 106 within bore 112 is secured to the interior wall of bore 112 by soldering or using an appropriate adhesive, for example an adhesive that is activated by ultraviolet light or heat. If hollow distal section 104 comprises nitinol, then an available special flux can be used to make a solder joint with shapeable member 106.

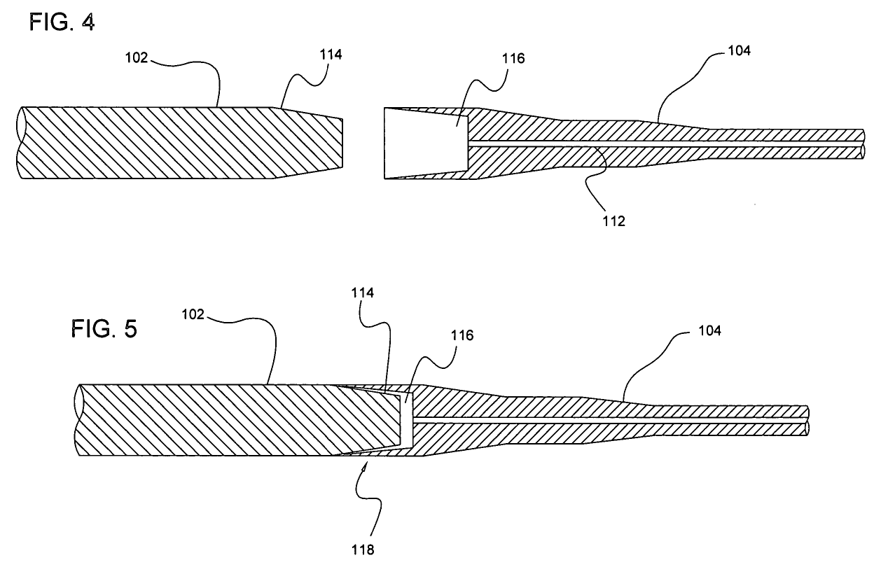

FIGS. 4 and 5 are longitudinal cross-sectional views of a portion of guidewire 100 that includes joint 118 between proximal section 102 and hollow distal section 104. In one embodiment of the invention, joint 118 is constructed by forming a narrowed or tapered portion 114 on the distal end of proximal section 102 and by forming a complementary recess 116 in the proximal end of distal section 104. The size and shape of recess 116 are selected so that tapered portion 114 may be inserted into the recess and form a close fit. Recess 116 may formed to the desired depth and shape by techniques such as mechanical drilling, laser drilling, or electrical discharge machining (EDM). The tapering or narrowing of portion 114 may be achieved, for example, by an abrasive technique such as centerless grinding.

As shown in FIG. 5, tapered portion 114 is inserted into recess 116 so that it fits tightly and forms a smooth exterior surface at the site of joint 118. Joint 118 is completed with suitable soldering or adhesive techniques. If either of sections 102, 104 comprises nitinol, then joint 118 may be formed by soldering with nitinol flux. In another embodiment, tapered portion 114 may be bonded to recess 116 with an adhesive.

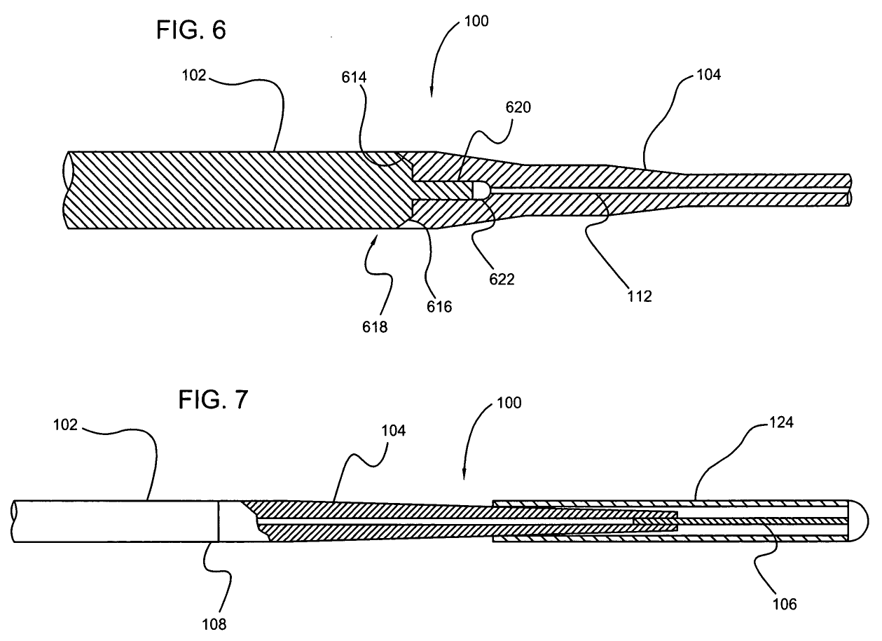

FIG. 6 illustrates alternative joint 618 between proximal section 102 and distal section 104 of guidewire 100. Comparable to the embodiment described above, narrowed portion 614 at the distal end of proximal section 102 is received within complementary recess 616 at the proximal end of distal section 104. In this alternative embodiment, stem 620 protrudes from narrowed portion 614 and is received within complementary socket 622. Socket 622 may be simply be the proximal end of bore 112, or it may be formed as part of, or a counterbore extending beyond recess 616. This embodiment of the invention avoids having an area at the proximal end of distal section 104 in which the wall is very thin, and potentially fragile, due to a tapered shape of the recess such as is shown in FIG. 4. Stem 620 and complementary socket 622 can also create a joint with higher tensile strength than a simple tapered joint as shown in FIGS. 4 and 5.

FIG. 7 is a partially sectioned illustration of guidewire 100 including flexible sleeve 124 disposed about shapeable member 106 and a reduced-diameter portion of distal section 104 to provide a substantially consistent diameter over the length of guidewire 100. Flexible sleeve 124 may be a metallic helical coil or a polymeric tube. In one embodiment of the invention, flexible sleeve 124 is a helical coil of stainless steel wire, and may also include a short distal coil segment of a more radiopaque wire. In other embodiments, flexible sleeve 124 comprises a polymeric material such as polyethylene, polyurethane, polytetrafluoroethylene (PTFE), or polyimide. Flexible sleeve 124 is designed to have a minimal effect on the flexibility of the region that it surrounds. At least the ends of flexible sleeve 124 are secured to distal section 104 and shapeable member 106 respectively, using appropriate adhesives or other methods of bonding such as soldering, with a nitinol flux, if required.

While the invention has been described with reference to particular embodiments, it will be understood by one skilled in the art that variations and modifications may be made in form and detail without departing from the spirit and scope of the invention.

1. An elongate medical guidewire comprising:
a proximal section and an adjoining hollow distal section, the distal section having a length and a wall thickness that decreases along at least a region of the length.

2. The guidewire of claim 1 wherein the wall thickness of the distal section decreases in a distal direction.

3. The guidewire of claim 1 wherein the distal section has:
a constant diameter inner bore extending there through; and
an outer diameter tapered to provide the decreasing wall thickness.

4. The guidewire of claim 3 wherein the diameter of the inner bore is between 0.002 and 0.003 inches.

5. The guidewire of claim 3 wherein the hollow distal section has been formed by selectively removing material from an outer surface of a heavy walled metal tube.

6. The guidewire of claim 1 wherein the proximal section comprises one or more materials selected from the group consisting of metals, metal alloys, stainless steel, nitinol, titanium alloys, nickel alloys, MP35N cobalt alloy, and tungsten alloys.

7. The guidewire of claim 1 wherein the hollow distal section comprises nitinol.

8. The guidewire of claim 1 further comprising a shapeable member coupled to a distal end of the hollow distal section.

9. The guidewire of claim 8 wherein a proximal end of the shapeable member is disposed within an inner bore extending through the hollow distal section.

10. The guidewire of claim 1 wherein a recess is formed in a proximal end of the distal section such that the configuration of the recess is complementary to a distal end of the proximal section.

11. The guidewire of claim 10 wherein the proximal end of the distal section is bonded to the distal end of the proximal section with an adhesive.

12. The guidewire of claim 10 wherein the proximal end of the distal section is soldered to the distal end of the proximal section.

13. The guidewire of claim 1 further comprising a flexible sleeve having a cylindrical outer surface and being disposed about at least a portion of the distal section, wherein the outer surface has a diameter consistent with a diameter of the proximal section.

14. A medical guidewire comprising:
an elongate member including a proximal section and a hollow distal section, the distal section including means for providing increased lateral flexibility along a length of the distal section.

15. The guidewire of claim 14 wherein the means for providing increased lateral flexibility comprises the hollow distal section having a wall thickness that is tapered.
